(19) United States
(12) Patent Application Publication
(10) Pub. No.: US 2026/0125335 A1
(43) Pub. Date: May 7, 2026

(54) SYNTHETIC CANNABINOID ANALOGS, PHARMACEUTICAL COMPOSITIONS, AND METHODS OF TREATING BACTERIAL INFECTIONS AND OTHER DISORDERS

(71) Applicant: Miralogx LLC, Tampa, FL (US)

(72) Inventor: Jonnie R. Williams, SR., Sarasota, FL (US)

(21) Appl. No.: 18/672,770

(22) Filed: May 23, 2024

Related U.S. Application Data
(60) Provisional application No. 63/524,381, filed on Jun. 30, 2023.
(60) Provisional application No. 63/623,564, filed on Jan. 22, 2024.

(51) Int. Cl. C07C 39/19; A61K 31/05; A61K 31/192; A61K 31/235; A61K 31/397; A61K 31/426; A61P 31/04; C07C 59/52; C07C 65/19; C07C 69/88; C07D 205/08; C07D 277/18

(52) U.S. Cl. CPC C07C 39/19; A61K 31/05; A61K 31/192; A61K 31/235; A61K 31/397; A61K 31/426; A61P 31/04; C07C 59/52; C07C 65/19; C07C 69/88; C07D 205/08; C07D 277/18; C07C 2601/10

(57) ABSTRACT

Cannabinoid analogs disclosed herein may exhibit antibacterial and anti-inflammatory properties. Pharmaceutical compositions comprising the cannabinoid analogs may be used to treat bacterial infections, including methicillin-resistant Staphylococcus aureus (MRSA) infections, as well as various disorders associated with chronic inflammation, such as arthritis. In some aspects, a pharmaceutical composition may be administered to an individual who has been exposed or is at risk of exposure to Bacillus anthracis or Bacillus anthracis spores.

### CROSS-REFERENCE TO RELATED APPLICATIONS

This application is a continuation-in-part of International Application No. PCT/US2022/050757, filed Nov. 22, 2022, which claims priority under 35 U.S.C. § 119(e) to U.S. App. No. 63/403,544, filed Sep. 2, 2022, and U.S. App. No. 63/283,431, filed Nov. 27, 2021. This application also claims priority under 35 U.S.C. § 119(e) to U.S. App. No. 63/524,381, filed Jun. 30, 2023, and U.S. App. No. 63/623,564, filed Jan. 22, 2024. Each of these applications is hereby incorporated by reference in its entirety.

### BACKGROUND

The surfacing of bacterial resistance to a number of antimicrobial agents such as beta-lactam antibiotics, macrolides, quinolones, and vancomycin has become a pervasive health problem. A significant problem in clinical practice is the increased incidence of methicillin-resistant Staphylococcus aureus (MRSA) infections. The mounting resistance of the important community acquired pathogen Streptococcus pneumoniae to penicillin and other antibacterial agents has become a global health problem. Multi drug-resistant strains of Mycobacterium tuberculosis have surfaced in several countries. The emergence and spread of resistant nosocomial and community-acquired pathogens has become a great menace to global public health.

Anthrax is an acute infectious disease caused by the spore-forming bacterium Bacillus anthracis. Anthrax most commonly occurs in wild and domestic lower vertebrates (e.g., cattle, sheep, goats, camels, antelopes, or other herbivores), but it can also occur in humans when they are exposed to infected animals or tissues from infected animals. In addition, Bacillus anthracis is one of the most important pathogens on the list of bioterrorism threats. The human LD50 for inhalational exposure is about 8,000 to 40,000 spores, or one deep breath at site of release.

Anthrax infection can occur in at least three forms-namely, inhalational, cutaneous, and gastrointestinal. Inhalation anthrax occurs in several discrete steps. Endospores of Bacillus anthracis are taken up by macrophages at the site of initial infection and can be transported to regional lymph nodes. The spores germinate inside the phagolysosome to become vegetative bacteria which can escape from the phagolysosome and replicate within the cytoplasm. Vegetative cells are released into the extracellular milieu and enter the circulation where the vegetative cells grow extracellularly to levels as high as 108 bacteria per ml of blood. In this environment, the vegetative bacteria respond to physiological body temperature and CO₂ levels to transcriptionally activate genes responsible for capsule formation and toxin synthesis. Finally, massive edema and organ failure are produced as a consequence of toxin formation. Experience with the 2001 bioterrorism incident found that once the disease reaches the phase where patients show evidence of significant toxin production, treatment with antibiotics can do little to prevent a fatal outcome. Similar results were reported in animal models. Accordingly, early diagnosis and intervention prior to toxin production is essential to patient survival.

Bacillus anthracis can also produce cutaneous anthrax or gastrointestinal anthrax. Cutaneous or gastrointestinal anthrax may show local signs and symptoms. In some cases, cutaneous or gastrointestinal anthrax can disseminate to produce the sepsis syndrome that occurs following inhalation anthrax.

Treatment of anthrax is dependent on administration of antibiotics early in the course of disease. Successful treatment requires that the bacterium be sensitive to available antibiotics and that antibiotics be administered before large amounts of toxin are released. A delay in antibiotic treatment may substantially lessen chances for survival. If a sufficient level of toxin production occurs, there is little in the way of specific therapy that is available for treatment.

A further complication in the treatment of anthrax is the possibility that a biowarfare strain can be engineered to resist treatment by conventional antibiotics. For example, there is a report of a Bacillus anthracis strain that has been engineered to resist the tetracycline and penicillin classes of antibiotics. Similarly, the bacillus could be engineered to produce a toxin that would evade anthrax vaccines that target the anthrax toxin.

Antibiotics have been increasingly investigated for their anti-inflammatory effects. In the setting of chronic rhinosinusitis, for example, macrolide and tetracycline antibiotics have been trialed for their anti-inflammatory properties. The anti-inflammatory mechanisms of macrolides include the downregulation of proinflammatory genes, improvement of mucociliary function, and decreased neutrophil accumulation. Observational studies provide support for a prolonged trial of macrolide therapy when conventional therapies fail, especially in patients with low serum IgE levels. Tetracyclines exert anti-inflammatory effects by decreasing inflammatory factors, decreasing neutrophil chemotaxis, and decreasing IgE production. Tetracyclines were shown in one study to decrease nasal polyp size but without any lasting symptom improvement. Other antibiotics shown to exhibit anti-inflammatory effects include trimethoprim-sulfamethoxazole and dapsone.

There remains a need for new compounds for treating patients infected with bacteria, particularly the multi drug-resistant bacteria such as MRSA and VRE. There also is an urgent need for new therapies to treat anthrax. It would be particularly desirable to develop new antibiotic compounds that also exhibit anti-inflammatory properties.

### SUMMARY

According to one aspect, a compound has a structure of Formula (I):

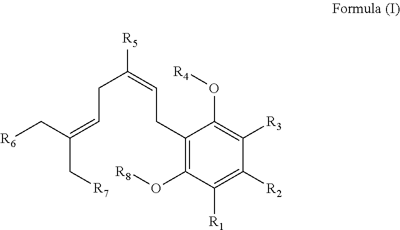

wherein R₁, R₂, R₃, R₄, R₅, R₆, R₇, and R₈ are each independently selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, —NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt, ester or ether thereof.

In another aspect, a method of treating a bacterial infection comprises administering to an individual in need thereof a pharmaceutical composition comprises a therapeutically effective amount of a compound of Formula (I), or a pharmaceutically acceptable salt, ester or ether thereof and a pharmaceutically acceptable vehicle therefor.

According to other aspects, a compound has a structure of Formula (II):

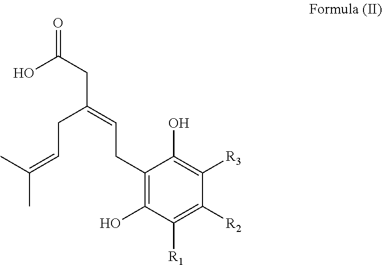

wherein R₁, R₂, and R₃ are as previously defined, or a pharmaceutically acceptable salt, ester or ether thereof.

In another aspect, a method of treating a bacterial infection comprises administering to an individual in need thereof a pharmaceutical composition comprises a therapeutically effective amount of a compound of Formula (II), or a pharmaceutically acceptable salt, ester or ether thereof and a pharmaceutically acceptable vehicle therefor.

According to another aspect, a compound has a structure of Formula (III):

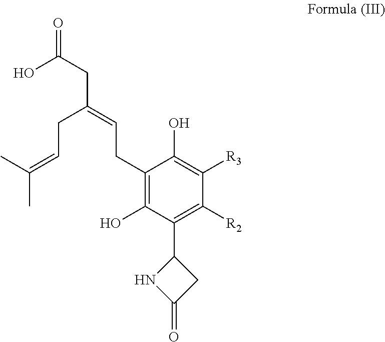

wherein R₂ and R₃ are as previously defined, or a pharmaceutically acceptable salt, ester, or ether thereof.

In another aspect, a method of treating a bacterial infection comprises administering to an individual in need thereof a pharmaceutical composition comprises a therapeutically effective amount of a compound of Formula (III), or a pharmaceutically acceptable salt, ester, or ether thereof and a pharmaceutically acceptable vehicle therefor.

According to other aspects, a compound has a structure of Formula (IV):

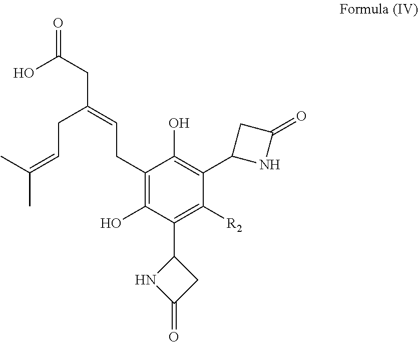

wherein R₂ is as previously defined, or a pharmaceutically acceptable salt, ester, or ether thereof.

In another aspect, a method of treating a bacterial infection comprises administering to an individual in need thereof a pharmaceutical composition comprises a therapeutically effective amount of a compound of Formula (IV), or a pharmaceutically acceptable salt, ester, or ether thereof and a pharmaceutically acceptable vehicle therefor.

According to another aspect, a compound has a structure of Formula (V):

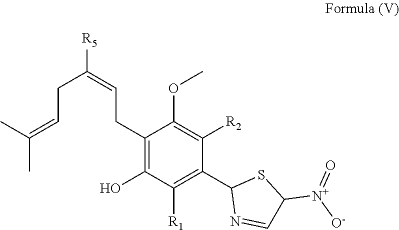

wherein R₁, R₃, and R₅ are as previously defined, or a pharmaceutically acceptable salt, ester, or ether thereof.

In another aspect, a method of treating a bacterial infection comprises administering to an individual in need thereof a pharmaceutical composition comprises a therapeutically effective amount of a compound of Formula (V), or a pharmaceutically acceptable salt, ester, or ether thereof and a pharmaceutically acceptable vehicle therefor.

According to yet another aspect, a compound has a structure of Formula (VI):

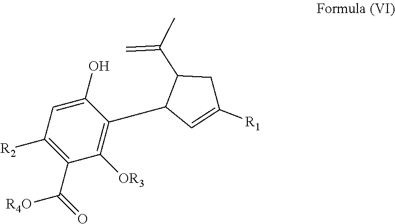

wherein R₁, R₂, R₃ and R₄ are as previously defined, or a pharmaceutically acceptable salt, ester, or ether thereof.

In another aspect, a method of treating a bacterial infection comprises administering to an individual in need thereof a pharmaceutical composition comprises a therapeutically effective amount of a compound of Formula (VI), or a pharmaceutically acceptable salt, ester, or ether thereof and a pharmaceutically acceptable vehicle therefor.

In still another aspect, a method of treating a viral infection comprises administering to an individual in need thereof a pharmaceutical composition comprises a therapeutically effective amount of a compound of Formula (VI), or a pharmaceutically acceptable salt, ester, or ether thereof and a pharmaceutically acceptable vehicle therefor.

In yet another aspect, a method of treating anxiety, depression or pain comprises administering to an individual in need thereof a pharmaceutical composition comprises a therapeutically effective amount of a compound of Formula (VI), or a pharmaceutically acceptable salt, ester, or ether thereof and a pharmaceutically acceptable vehicle therefor.

In some embodiments, a compound has a structure selected from the group consisting of:

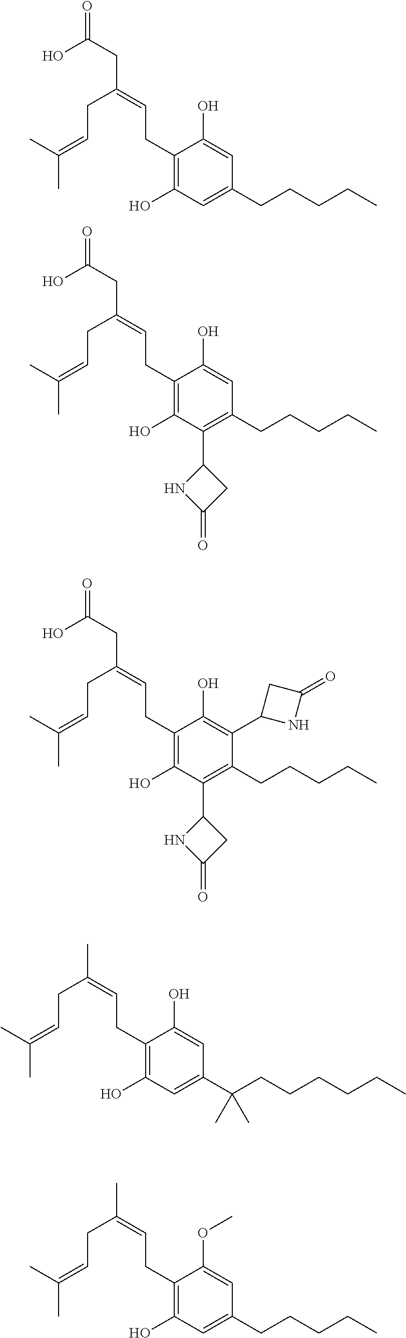

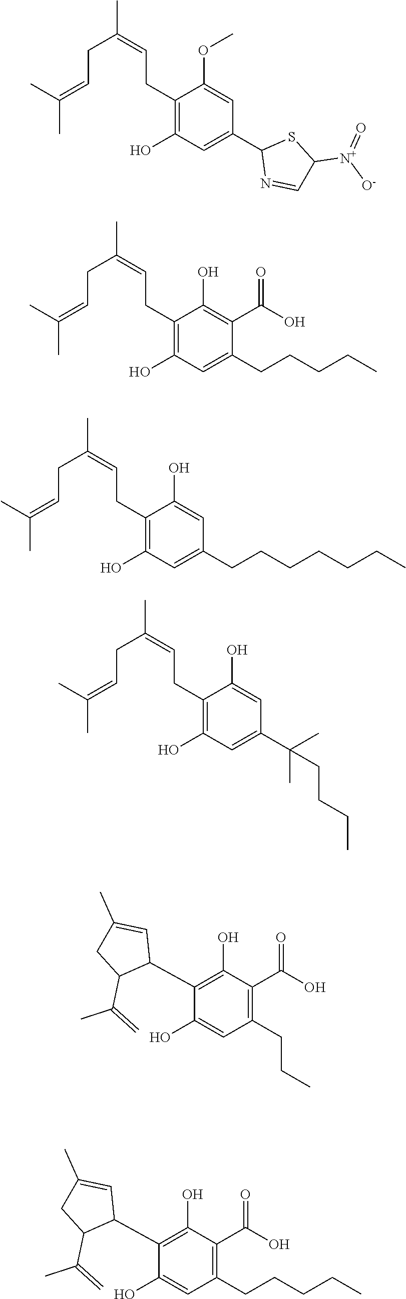

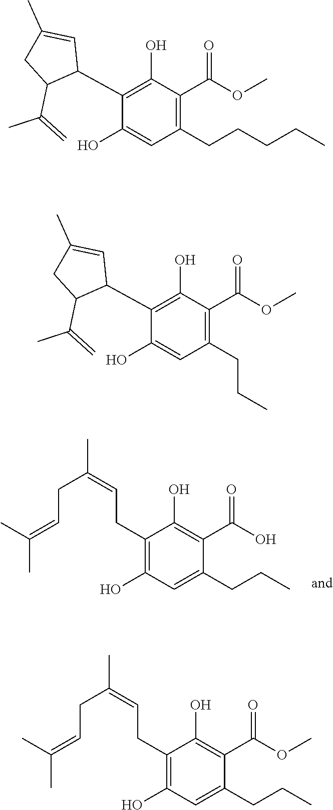

or a pharmaceutically acceptable salt, ester or ether thereof.

### DETAILED DESCRIPTION

Cannabinoids produced by the Cannabis sativa plant have the potential to treat a vast assortment of diseases and other human ailments. More than 100 different cannabinoids have been isolated from cannabis and each cannabinoid compound exhibits various effects. For example, THC is well-known for its psychological effects and CBD is known for its non-psychoactive effects. THC and related derivatives typically exert therapeutic activities via cannabinoid receptors found in humans and other mammals. CBD is an isomer of THC. CBD and CBD derivatives also exhibit anti-oxidative and anti-inflammatory effects through pathways not related to cannabinoid receptors. Cannabinoid type 1 (CB₁) receptors are found primarily in the brain, including the basal ganglia and in the limbic system, and the hippocampus and the striatum, as well as the cerebellum. CB₁ receptors can be found in the human anterior eye and retina. Research indicates that cannabinoid type 2 (CB₂) receptors are responsible for anti-inflammatory and other therapeutic effects related to cannabinoids.

Cannabis plants that contain high levels of cannabinoids such as THC, for example, are typically known as “marijuana” plants. Cannabis plants with a low cannabinoid content are categorized as “hemp” plants. Individual countries usually determine the levels of cannabinoids that differentiate between cannabis plants that are categorized as marijuana or hemp plants. Generally, the THC content on a dry-weight basis for a cannabis plant categorized as a hemp plant is 0.3% or less. Cannabis sativa plants having THC, CBD, and other cannabinoid content levels greater than 0.3% are typically considered marijuana plants. Medical marijuana typically contains cannabinoid levels between 5 and 20%. Other Cannabis sativa plants may produce cannabinoid levels from 25 to 30%.

The biosynthetic pathway of the Cannabis sativa plant that produces the various cannabinoids starts with the precursor cannabigerolic acid. The enzymes THCA synthase and CBDA synthase catalyze the biosynthesis of cannabigerolic acid to tetrahydrocannabinol acid (THCA) and cannabidiol acid (CBDA), respectively, as well as other cannabinoids. It is known that various other cannabinoids are produced via this pathway. THC, CBD, and other cannabinoid derivatives are generated artificially from THCA and CBDA by non-enzymatic decarboxylation. Aizpurua-Olaizola et al., “Evolution of the Cannabinoid and Terpene Content during the Growth of Cannabis sativa Plants from Different Chemotypes,” J. Natural Prods. 2016 79 (2), 324-331. Various classes of cannabinoids are biosynthesized via this general pathway to include THC (tetrahydrocannabinol), THCA (tetrahydrocannabinolic acid), CBD (cannabidiol), CBDA (cannabidiolic Acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran).

Cannabigerol (CBG) is known to inhibit the proliferation of gram-positive facultatively anaerobic bacterium. Bacterium treated with CBG becomes swollen and has altered membrane structures by inducing membrane hyperpolarization and decreasing membrane fluidity. Calcein AM staining suggests that CBG increases membrane permeability and may further reduce metabolic activity.

CBG also has been identified as exerting anti-biofilm activity, as demonstrated by reduced metabolic activity observed after treating preformed biofilms with CBG. CBG also may prevent pH drops that correlate to reduced bacterial growth. One study found that CBG reduces the expression of essential bio-film regulating genes, prevents exopolysaccharide (EPS) production, inhibits quorum sensing, and increases reactive oxygen species (ROS) production. M. Aqawi et al., The Antimicrobial Activity of Cannabinoids, Antibiotics 2020, 9, 406.

### I. Synthetic Cannabinoid Analogs

According to one aspect, a compound has a structure of Formula (I):

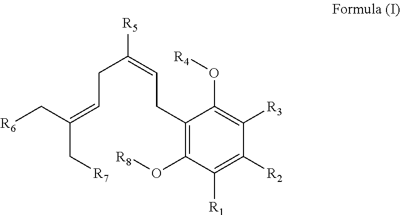

wherein R₁, R₂, R₃, R₄, R₅, R₆, R₇, R₈ are each independently selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, —NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COH, —C(O)C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt, ester or ether thereof.

According to other aspects, a compound has a structure of Formula (II):

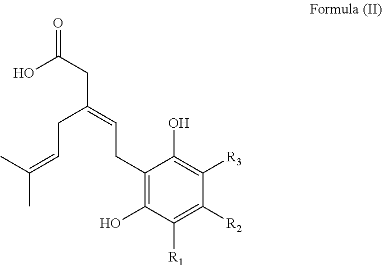

wherein R₁, R₂, and R₃ are as previously defined, or a pharmaceutically acceptable salt, ester or ether thereof.

According to another aspect, a compound has a structure of Formula (III):

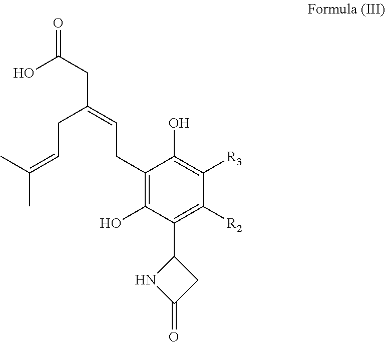

wherein R₂ and R₃ are as previously defined, or a pharmaceutically acceptable salt, ester or ether thereof.

According to other aspects, a compound has a structure of Formula (IV):

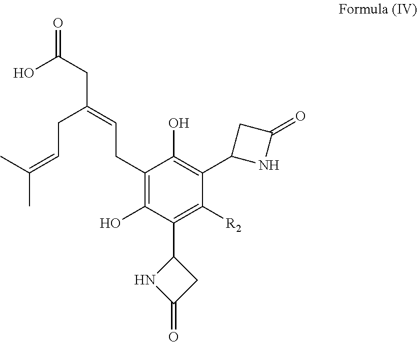

wherein R₂ is as previously defined, or a pharmaceutically acceptable salt, ester or ether thereof.

According to another aspect, a compound has a structure of Formula (V):

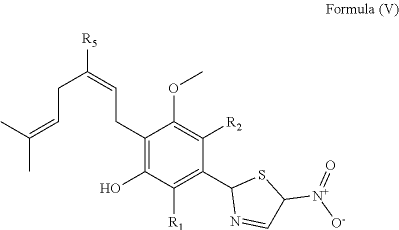

wherein R₁, R₃, and R₅ are as previously defined, or a pharmaceutically acceptable salt, ester or ether thereof.

According to yet another aspect, a compound has a structure of Formula (VI):

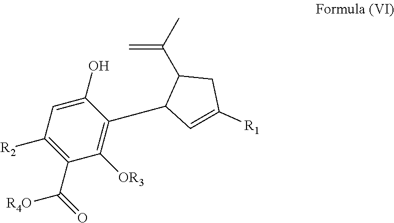

wherein R₁, R₂, R₃ and R₄ are as previously defined, or a pharmaceutically acceptable salt, ester, or ether thereof.

In some examples of Formula (VI) compounds, R₁ and R₃ are each H, and R₂ is alkyl, such as C₃₋₉ straight chained or branched alkyl, e.g., n-propyl, n-butyl, n-pentyl, or 2,2-dimethylheptyl.

In some examples of Formula (VI) compounds, R₄ is H. In other examples of Formula (VI) compounds, R₄ is alkyl, such as C₁₋₃ alkyl, e.g., methyl.

In another aspect, a method of treating a bacterial infection comprises administering to an individual in need thereof a pharmaceutical composition comprises a therapeutically effective amount of a compound of Formula (VI), or a pharmaceutically acceptable salt, ester, or ether thereof and a pharmaceutically acceptable vehicle therefor.

In still another aspect, a method of treating a viral infection comprises administering to an individual in need thereof a pharmaceutical composition comprises a therapeutically effective amount of a compound of Formula (VI), or a pharmaceutically acceptable salt, ester, or ether thereof and a pharmaceutically acceptable vehicle therefor.

In yet another aspect, a method of treating anxiety, depression or pain comprises administering to an individual in need thereof a pharmaceutical composition comprises a therapeutically effective amount of a compound of Formula (VI), or a pharmaceutically acceptable salt, ester, or ether thereof and a pharmaceutically acceptable vehicle therefor.

In some embodiments, a compound has a structure selected from the group consisting of:

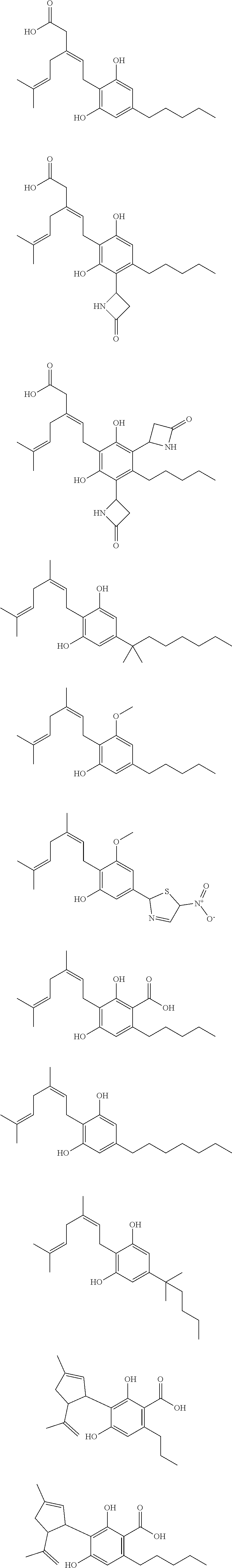

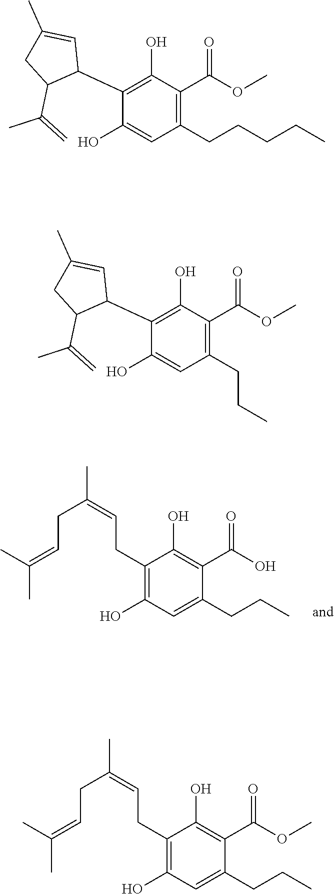

or a pharmaceutically acceptable salt, ester or ether thereof.

As used herein the term “alkyl,” whether alone or as part of a substituent group, refers to a saturated C₁-C₁ carbon chain, wherein the carbon chain may be straight or branched; wherein n can be 2, 3, 4, 5, 6, 7, 8, 9 or 10. Suitable examples include, but are not limited to methyl, ethyl, propyl, isopropyl, butyl, isobutyl, sec-butyl, t-butyl, n-pentyl and n-hexyl.

As used herein the term “alkenyl,” whether alone or as part of a substituent group, refers to a C₂-Cₙ carbon chain, wherein the carbon chain may be straight or branched, wherein the carbon chain contains at least one carbon-carbon double bond, and wherein n can be 3, 4, 5, 6, 7, 8, 9 or 10.

As used herein the term “alkynyl,” whether alone or as part of a substituent group, refers to a C₂-Cₙ, wherein the carbon chain may be straight or branched, wherein the carbon chain contains at least one carbon-carbon triple bond, and wherein n can be 3, 4, 5, 6, 7, 8, 9 or 10.

As used herein the term “aryl,” whether alone or as part of a substituent group, refers to an unsubstituted carboxylic aromatic ring comprising between 6 to 14 carbon atoms. Suitable examples include, but are not limited to, phenyl and naphthyl.

As used herein the term “protected hydroxyl” refers to a hydroxyl group substituted with a suitably selected oxygen protecting group. More particularly, a “protected hydroxyl” refers to a substituent group of the formula —OPG₁ wherein PG₁ is a suitably selected oxygen protecting group. During any of the processes for preparation of the compounds of the present disclosure it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein the term “oxygen protecting group” refers to a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM and THP. Other suitable oxygen protecting groups may be found in texts such as T. W. Greene & P. G. M. Wuts, Protective Groups in Organic Synthesis, John Wiley & Sons, 1991.

As used herein the term “nitrogen protecting group” refers to a group which may be attached to a nitrogen atom to protect said nitrogen atom from participating in a reaction and which may be readily removed following the reaction. Suitable nitrogen protecting groups include, but are not limited to, carbamates groups of the formula C(O)—OR wherein R can be methyl, ethyl, t-butyl, benzyl, phenylethyl, CH₂═CH—CH₂—, and the like; amide groups of the formula —C(O)—R′ wherein R′ can be methyl, phenyl, trifluoromethyl, and the like; N-sulfonyl derivative groups of the formula —SO₂—R″ wherein R″ can be tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein the term “acyl” refers to a group of the formula —CO—Cₙ wherein Cₙ represent a straight or branched alkyl chain wherein n can be 1, 2, 3, 4, 5, 6, 7, 8, 9 or 10.

As used herein the term “heteroaryl” refers to any five or six membered monocyclic aromatic ring structure containing at least one heteroatom selected from the group consisting of O, N and S, and optionally containing one to three additional heteroatoms independently selected from the group consisting of O, N and S; or a nine or ten membered bicyclic aromatic ring structure containing at least one heteroatom selected from the group consisting of O, N and S, and optionally containing one to four additional heteroatoms independently selected from the group consisting of O, N and S. The heteroaryl group may be attached at any heteroatom or carbon atom of the ring such that the result is a stable structure. Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl and pteridinyl.

As used herein the term “cycloalkyl” refers to any monocyclic ring containing from four to six carbon atoms, or a bicyclic ring containing from eight to ten carbon atoms. The cycloalkyl group may be attached at any carbon atom of the ring such that the result is a stable structure. Examples of suitable cycloalkyl groups include, but are not limited to, cyclopropyl, cyclobutyl, cyclopentyl and cyclohexyl.

As used herein the term “heterocycle” refers to any four to six membered monocyclic ring structure containing at least one heteroatom selected from the group consisting of O, N and S, and optionally containing one to three additional heteroatoms independently selected from the group consisting of O, N and S; or an eight to ten membered bicyclic ring structure containing at least one heteroatom selected from the group consisting of O, N and S, and optionally containing one to four additional heteroatoms independently selected from the group consisting of O, N and S. The heterocycle group may be attached at any heteroatom or carbon atom of the ring such that the result is a stable structure. Examples of suitable heterocycle groups include, but are not limited to, azetidine, azete, oxetane, oxete, thietane, thiete, diazetidine, diazete, dioxetane, dioxete, dithietane, dithiete, pyrrolidine, pyrrole, tetrahydrofuran, furan, thiolane, thiophene, piperidine, oxane, thiane, pyridine, pyran and thiopyran.

The groups described herein can be unsubstituted or substituted, as herein defined. In addition, the substituted groups can be substituted with one or more groups such as a C₁-C₆ alkyl, C₁₋₄ alkyl, —O—C₁₋₄ alkyl, hydroxyl, amino, (C₁₋₄ alkyl)amino, di(C₁₋₄ alkyl)amino, —S—(C₁₋₄ alkyl), —SO—(C₁₋₄ alkyl), —SO₂—(C₁₋₄ alkyl), halogen, aryl, heteroaryl, and the like.

With reference to substituents, the term “independently” means that when more than one of such substituents is possible, such substituents may be the same or different from each other.

The compounds of the present disclosure may contain at least one hydroxyl group. These at least one hydroxyl group may form an ester with inorganic or organic acid. In particular, pharmaceutically acceptable acids. The ester(s) may form chiral carbons. The present disclosure is directed toward all stereo-chemical forms of the compounds of the present disclosure, including those formed by the formation of one or more ester groups.

### II. Synthesis and Purification of the Cannabinoid Compounds

In some examples, the cannabinoid compounds described herein may be formed as salts, which may be helpful to improve chemical purity, stability, solubility, and/or bioavailability. Non-limiting examples of possible salts are described in P. H. Stahl et al., Handbook of Pharmaceutical Salts: Properties, Selection and Use, Weinheim/Zürich:Wiley-VCH/VHCA, 2002, including salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

The compounds described herein may be prepared synthetically using known techniques with appropriate modifications to the reactants to form the structures shown herein or by other suitable pathways that will be apparent to persons skilled in the art. By way of non-limiting example, compounds described herein may be synthesized according to one or more of the following pathways described in Razdan, Total Synthesis of Cannabinoids, SISA Incorporated, Cambridge, Massachusetts, with appropriate modifications to the reactants, as will be apparent to persons skilled in the art, to yield the structures disclosed herein. Alternatively, the synthesis techniques described in Smeltzer et al. WO 2020/077153 A1, the disclosure of which is hereby incorporated by reference in its entirety, may be suitably adapted to synthesize the cannabigerol analogs described herein.

Compounds intended for administration to humans or other mammals generally should have very high purity. In the case of synthetically prepared compounds, purity refers to the ratio of a compound's mass to the total sample mass following any purification steps. Usually, the level of purity is at least about 95%, more usually at least about 96%, about 97%, about 98%, or higher. For example, the level of purity may be about 98.5%, 99.0%, 99.1%, 99.2%, 99.3%, 99.4%, 99.5%, 99.6%, 99.7%, 99.8%, 99.9%, or higher.

Compounds described herein that exist in more than one optical isomer form (enantiomers) may be provided either as racemic mixture or by isolating one of the enantiomers, the latter case in which purity as described above may refer to enantiomeric purity.

### III. Methods of Use

The compounds as described herein are particularly useful as antibacterial agents. The compounds may be used, for example, in the treatment of bacterial infections caused by bacteria belonging to Staphylococcus, Streptococcus, Enterococcus or Bacillus species. Staphylococcus species refers to a Gram-positive bacteria, which appears as grape-like clusters when viewed through a microscope and as large, round, golden-yellow colonies, often with β-hemolysis, when grown on blood agar plates. Staphylococcus aureus which belongs to Staphylococcus species causes a variety of suppurative (pus-forming) infections such as superficial skin lesions such as boils, styes and furunculosis; more serious infections such as pneumonia, mastitis, phlebitis, meningitis, and urinary tract infections; and deep-seated infections, such as osteomyelitis and endocarditis. Staphylococcus aureus is a major cause of hospital acquired (nosocomial) infection of surgical wounds and infections associated with indwelling medical devices. Staphylococcus aureus causes food poisoning by releasing enterotoxins into food, and toxic shock syndrome by release of superantigens into the blood stream.

In some examples, a compound disclosed herein may be administered to an individual in need thereof for treating a methicillin-resistant Staphylococcus aureus (MRSA) infection.

Streptococcus species refers to a genus of spherical, Gram-positive bacteria, and a member of the phylum Firmicutes. Streptococci are lactic acid bacteria. Streptococcus species are responsible for infectious diseases such as meningitis, bacterial pneumonia, endocarditis, erysipelas and necrotizing fasciitis (the ‘flesh-eating’ bacterial infections).

Enterococcus species refers to a genus of lactic acid bacteria of the phylum Firmicutes. They are Gram-positive cocci which often occur in pairs (diplococci). Enterococci are facultative anaerobic organisms. Enterococci are among the most frequent causes of hospital-acquired infections. Enterococci develop resistance to antibiotics such as gentamicin and vancomycin.

Bacillus species refers to a large number of diverse, rod-shaped Gram positive bacteria that are motile by peritrichous flagella and are aerobic. It is also a member of the division Firmicutes. Members of this genus are capable of producing endospores that are highly resistant to unfavorable environment conditions. Bacillus cereus which belongs to Bacillus species causes two types of food-borne intoxications. One type is characterized by the symptoms of nausea, vomiting and abdominal cramps. The second type is manifested primarily by abdominal cramps and diarrhea. Infections attributed to Bacillus subtilis which belongs to Bacillus species, include bacteremia, endocarditis, pneumonia, and septicemia in patients in compromised immune states.

A compound disclosed herein may be administered prophylactically for immunizing a subject against anthrax or other infectious diseases. For example, a compound may be administered to a human who has been exposed or is at risk of exposure to Bacillus anthracis or Bacillus anthracis spores. Such administration may be made, for example, to military or security force personnel, mail handlers, clinicians or laboratorians who may have close contact with Bacillus anthracis spores, civilians or industrial workers who may be exposed to infected animals or their products, newborns or children, elderlies, or the public at large. In some examples, a compound disclosed herein may be administered post-exposure for preventing the development of serious disease conditions.

In some aspects, compounds described herein may be administered for treating a viral infection. Viruses gain infamy through a combination of large infection rates and death, even when their strength has been relatively subdued. Rabies, measles and chickenpox are still notorious, even though vaccines and medications have drastically reduced their deadliness. Some viruses are either no longer a threat or not perceived as threatening. For example, smallpox has been eradicated, and there has not been a polio case of American origin since 1979.

Other viruses are still active and pose a serious threat to health. Viruses including coronaviruses (e.g., COVID-19), hepatitis, HIV, and influenza still cause mass infection and significant death rates. COVID-19 is transmitted through respiratory droplets and uses receptor-mediated entry into a human host via angiotensin-converting enzyme II (ACE2) that is expressed in lung tissue, as well as oral and nasal mucosa, kidney, testes, and the gastrointestinal tract. Modulation of ACE2 levels in these gateway tissues may decrease disease susceptibility. See Wang et al., In Search of Preventative Strategies: Novel Anti-Inflammatory High-CBD Cannabis sativa Extracts Modulate ACE2 Expression in COVID-19 Gateway Tissues (Apr. 17, 2020), doi: 10.20944/preprints202004.0315.v1. The cannabinoid analogs as described herein may modulate ACE2 expression and may have utility in the treatment of a coronavirus such as COVID-19.

There are three different virus types that are made distinct by their shape. The cylindrical helical virus type is associated with the tobacco mosaic virus. Envelope viruses, such as influenza and HIV, come covered in a protective lipid envelope. Most animal viruses are classified as icosahedral and are nearly spherical in shape.

The viruses within these categories share similar characteristics. They are made up of RNA or DNA, and are coated with either a protein, lipid or glycoprotein. They also are parasitic, meaning they cannot replicate without a host. Viruses are also the most abundant biological form of life on the planet. While they cannot be cured, a vaccination can be effective to prevent their spread.

In one example, the virus is a human immunodeficiency virus (HIV), hepatitis, coronavirus (e.g., COVID-19), influenza, or the common cold. In another example, the virus is herpes simplex virus (HSV) or human papillomavirus (HPV). Low-risk mucosal HPVs such as HPV-6 and HPV-11 cause genital warts (condyloma accuminata), whereas the high-risk HPVs cause squamous intraepithelial lesions that can progress to invasive squamous cell carcinoma. The vast majority of human cervical cancers are associated with high-risk HPV infections. HPV-16 is by far the most prevalent mucosal high-risk HPV type, followed by HPV-18 and HPV-31. Approximately 20% of oral cancers, particularly oropharyngeal carcinomas in patients that lack the classical risk factors of tobacco and alcohol abuse, are also high-risk HPV positive. Other anogenital tract malignancies that are also frequently associated with high-risk HPV infections include penile and vulvovaginal cancers as well as anal carcinomas, which frequently occur in individuals with human immunodeficiency virus (HIV)-associated AIDS.

The compounds disclosed herein may have anti-inflammatory activity. For example, a compound may have an anti-inflammatory activity capable of reducing the levels of an inflammation-inducing molecule. The disclosed compounds may have an anti-inflammatory activity capable of reducing the levels of substance P(SP), calcitonin gene-related peptide (CGRP), glutamate, or a combination thereof. A compound may have an anti-inflammatory activity capable of reducing the levels of SP, CGRP, glutamate, or a combination thereof released from a sensory neuron by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%.

Prostaglandins mediate a local inflammatory response and are involved in all inflammatory functions through action on prostaglandin receptors and mediate inflammatory signaling including chemotaxis (macrophages, neutrophils and eosinophils), vasodilation and algesia. However, the PG-mediated inflammatory response is self-limiting (resolving). The principle resolution factor is a prostaglandin called 15dPGJ2, which is an endogenous agonist of peroxisome proliferator-activator receptor-γ (PPAR-γ) signaling. PPAR-γ signaling pathway 1) induces apoptosis of macrophage M1 cells, thereby reducing the levels of Th1 pro-inflammatory cytokines and 2) promotes differentiation of monocytes into macrophage M2 cells. Macrophage M2 cells produce and release Th2 anti-inflammatory cytokines.

Compounds disclosed herein may have an anti-inflammatory activity capable of reducing the levels of an inflammation inducing prostaglandin. A compound may have an anti-inflammatory activity capable of reducing the levels of an inflammation inducing prostaglandin released from a sensory neuron by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. A compound may have an anti-inflammatory activity capable of reducing the levels of an inflammation inducing prostaglandin released from a sensory neuron in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

The peroxisome proliferator-activated receptors (PPARs) are a group of nuclear receptor proteins that function as transcription factors regulating the expression of genes. All PPARs are known to heterodimerize with the retinoid X receptor (RXR) and bind to specific regions on the DNA of target genes called peroxisome proliferator hormone response elements (PPREs). PPARs play essential roles in the regulation of cellular differentiation, development, and metabolism (carbohydrate, lipid, protein), and tumorigenesis of higher organisms. The family comprises three members, PPAR-α, PPAR-γ, and PPAR-δ (also known as PPAR-β). PPAR-α is expressed in liver, kidney, heart, muscle, adipose tissue, as well as other tissues. PPAR-δ is expressed in many tissues but markedly in brain, adipose tissue, and skin. PPAR-γ comprises three alternatively-spliced forms, each with a different expression pattern. PPAR-γ1 is expressed in virtually all tissues, including heart, muscle, colon, kidney, pancreas, and spleen. PPAR-γ2 is expressed mainly in adipose tissue. PPAR-γ3 is expressed in macrophages, large intestine, and white adipose tissue. Endogenous ligands for the PPARs include free fatty acids and eicosanoids. PPAR-7 is activated by PGD2 (a prostaglandin), whereas PPAR-α is activated by leukotriene B4.

A compound may have an anti-inflammatory activity capable of reducing the levels of IFN-γ, TNF-α, IL-12, or a combination thereof released from a Th1 cell and increasing the levels of IL-10 released from a Th2 cell. A compound may have an anti-inflammatory activity capable of reducing the levels of IFN-γ, TNF-α, IL-12, or a combination thereof released from a Th1 cell by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, and capable of increasing the levels of IL-10 released from a Th2 cell by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%.

A compound may have an anti-inflammatory activity capable of stimulating some or all PPAR signaling pathways. It is contemplated that such a compound therefore may act as a PPAR pan-agonist or possibly as a selective PPAR agonist.

A compound may have an anti-inflammatory activity capable of modulating Th1 and Th2 cytokines. A compound may have an anti-inflammatory activity capable of reducing the levels of Interferon-γ (IFN-γ), tumor necrosis factor-α (TNF-α), interleukin-12 (IL-12), or a combination thereof released from a Th1 cell. A compound may have an anti-inflammatory activity capable of reducing the levels of IFN-γ, TNF-α, IL-12, or a combination thereof released from a Th1 cell by, e.g., at least 10%, at least 20%, at least 30%, at least 40%, at least 50%, at least 60%, at least 70%, at least 80%, or at least 90%. A compound may have an anti-inflammatory activity capable of reducing the levels of IFN-γ, TNF-α, IL-12, or a combination thereof released from a Th1 cell in a range from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, or about 10% to about 90.

A compound may have an anti-inflammatory activity capable of increasing the levels of IL-10 released from a Th2 cell. A compound may have an anti-inflammatory activity capable of increasing the levels of IL-10 released from a Th2 cell by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%.

In some aspects, a cannabinoid analog as described herein is administered to an individual in need thereof for the treatment of a substance addiction, such as alcohol, tobacco, opioid, prescription drugs, cocaine, benzodiazepines, amphetamines, hallucinogens, inhalants, phencyclidine, or other drug addictions. Such treatments also are inclusive of treating withdrawal in dependency on benzodiazepines, opiates, or alcohol, as well as symptoms experienced by patients with substance use disorders, such as anxiety, mood symptoms, pain, and insomnia.

In addition to anxiety that is associated with substance use disorders, the cannabinoid analogs may be effective for treating other types of anxiety disorders, such as post-traumatic stress disorder, general anxiety disorder, panic disorder, social anxiety disorder, and obsessive-compulsive disorder.

In other aspects, a cannabinoid analog as described herein may be administered to an individual in need thereof for the treatment of multiple sclerosis, fibromyalgia, epilepsy or neuropsychiatric disorders that are linked to epilepsy, such as neurodegeneration, neuronal injury, and psychiatric diseases. The cannabinoid analogs may be effective for potentiating the anticonvulsant activity of other active agents such as phenytoin and diazepam.

In still other aspects, a cannabinoid analog as described herein may be used in as an antipsychotic for treating patients with schizophrenia. The cannabinoid analogs also may be effective to reduce intraocular pressure, such as in the treatment of glaucoma.

In yet other aspects, a cannabinoid analog as described herein may be administered to an individual in need thereof for the treatment of cancer. The cannabinoid analog may be effective to block cancer cells from spreading around the body and invading an area entirely; for suppressing the growth of cancer cells and/or promoting the death of cancer cells.

The cannabinoid analogs as described herein may be useful in the treatment of Type 1 diabetes, which is caused by inflammation when the immune system attacks cells in the pancreas; as well as acne, which is caused, in part, by inflammation and overworked sebaceous glands on the body. The anti-inflammatory properties of the compounds may lower the production of sebum that leads to acne, including acne vulgaris, the most common form of acne.

The cannabinoid analogs as described herein may be used to treat Alzheimer's disease, and particularly to prevent the development of social recognition deficit in subjects when administered in the early stages of Alzheimer's disease. Other examples of disorders that may be treated by the cannabinoid analog as described herein include nausea, vomiting, anorexia, and cachexia. The compounds may produce an appetite-enhancing effect, for example in AIDS patients or individuals with Alzheimer's disease who refuse food.

The cannabinoid analogs as described herein may be useful in the treatment of spasticity caused by multiple sclerosis (MS) or spinal cord injury, movement disorders, such as Tourette's syndrome, dystonia, or tardive dyskinesia. MS patients may experience benefits on ataxia and reduction of tremors.

Analgesic properties of the cannabinoid analogs may prove beneficial, for example, in the treatment of neuropathic pain due to multiple sclerosis, damage of the brachial plexus and HIV infection, pain in rheumatoid arthritis, cancer pain, headache, menstrual pain, chronic bowel inflammation and neuralgias.

The cannabinoid analogs as described herein may be useful in the treatment of asthma. Experiments examining the anti-asthmatic effect of THC or cannabis date mainly from the 1970s, and are all acute studies. The effects of a cannabis cigarette (2% THC) or oral THC (15 mg), respectively, approximately correspond to those obtained with therapeutic doses of common bronchodilator drugs (salbutamol, isoprenaline). Since inhalation of cannabis products may irritate the mucous membranes, oral administration or another alternative delivery system would be preferable. Very few patients developed bronchoconstriction after inhalation of THC.

An improvement of mood in reactive depression has been observed in several clinical studies with THC. There are additional case reports claiming benefit of cannabinoids in other psychiatric symptoms and diseases, such as sleep disorders, anxiety disorders, bipolar disorders, and dysthymia. Various authors have expressed different viewpoints concerning psychiatric syndromes and cannabis. While some emphasize the problems caused by cannabis, others promote the therapeutic possibilities. Quite possibly cannabis products may be either beneficial or harmful, depending on the particular case. The attending physician and the patient should be open to a critical examination of the topic, and a frankness to both possibilities.

In a number of painful syndromes secondary to inflammatory processes (e.g. ulcerative colitis, arthritis), cannabis products may act not only as analgesics but also demonstrate anti-inflammatory potential. For example, some patients employing cannabis report a decrease in their need for steroidal and nonsteroidal anti-inflammatory drugs. Moreover there are some reports of positive effects of cannabis self-medication in allergic conditions. It is as yet unclear whether cannabis products may have relevant effects on causative processes of autoimmune diseases.

There are a number of positive patient reports on medical conditions that cannot be easily assigned to the above categories, such as pruritus, hiccup, ADS (attention deficit syndrome), high blood pressure, tinnitus, chronic fatigue syndrome, restless leg syndrome, and others. Different authors have described several hundred possible indications for cannabis and THC. For example, 2.5 to 5 mg THC were effective in three patients with pruritus due to liver diseases. Another example is the successful treatment of a chronic hiccup that developed after a surgery. No medication was effective, but smoking of a cannabis cigarette completely abolished the symptoms.

Cannabis products often show very good effects in diseases with multiple symptoms that encompassed within the spectrum of THC effects, for example, in painful conditions that have an inflammatory origin (e.g., arthritis), or are accompanied by increased muscle tone (e.g., menstrual cramps, spinal cord injury), or in diseases with nausea and anorexia accompanied by pain, anxiety and depression, respectively (e.g. AIDS, cancer, hepatitis C).

Pharmaceutical compositions may be administered by any suitable route. For example, the compositions may be administered orally, parenterally, by inhalation spray, topically, rectally, nasally, buccally, vaginally, via an implanted reservoir, or ingested as a dietary supplement or food. In some embodiments, a composition is provided in an inhaler, which may be actuated to administer a vaporized medium that is inhaled into the lungs. The term parenteral as used herein includes subcutaneous, intracutaneous, intravenous, intramuscular, and intracranial injection or infusion techniques. Most often, the pharmaceutical compositions are readily administered orally and ingested.

Pharmaceutical compositions may contain any conventional non-toxic pharmaceutically-acceptable carriers, adjuvants or vehicles. In some cases, the pH of the formulation may be adjusted with acceptable pharmaceutical or food grade acids, bases or buffers to enhance the stability of the formulated composition or its delivery form.

Liquid dosage forms for oral administration include acceptable pharmaceutical or food grade emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylsulfoxide (DMSO) dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, lozenges, pills, powders, and granules. In such solid dosage forms, the active compound is mixed with at least one inert, acceptable pharmaceutical or food grade excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidone, sucrose and acacia, c) humectants such as glycerol, d) disintegrating agents such as agaragar, calcium carbonate, potato or tapioca starch, alginic acid, certain silicates, and sodium carbonate, e) solution retarding agents such as paraffin, f) absorption accelerators such as quaternary ammonium compounds, g) wetting agents such as cetyl alcohol and glycerol monostearate, h) absorbents such as kaolin and bentonite clay, i) lubricants such as talc, calcium stearate, magnesium stearate, solid polyethylene glycols, sodium lauryl sulfate, and mixtures thereof, and j) sweetening, flavoring, perfuming agents, and mixtures thereof. In the case of capsules, lozenges, tablets and pills, the dosage form may also comprise buffering agents.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract or, optionally, in a delayed or extended manner. Examples of embedding compositions which can be used include polymeric substances and waxes. Tablet formulations for extended release are also described in U.S. Pat. No. 5,942,244.

Compositions may contain a cannabinoid analog or compounds, alone or with other therapeutic compound(s). A therapeutic compound is a compound that provides pharmacological activity or other direct effect in the diagnosis, cure, mitigation, treatment, or prevention of disease, or to affect the structure or any function of the body of man or animals. A therapeutic compound disclosed herein may be used in the form of a pharmaceutically acceptable salt, solvate, or solvate of a salt, e.g., a hydrochloride. Additionally, therapeutic compound disclosed herein may be provided as racemates, or as individual enantiomers, including the R- or S-enantiomer. Thus, the therapeutic compound disclosed herein may comprise a R-enantiomer only, a S-enantiomer only, or a combination of both a R-enantiomer and a S-enantiomer of a therapeutic compound. In some aspects, the therapeutic compound may have anti-inflammatory activity, such as a non-steroidal anti-inflammatory drug (NSAID). NSAIDs are a large group of therapeutic compounds with analgesic, anti-inflammatory, and anti-pyretic properties. NSAIDs reduce inflammation by blocking cyclooxygenase. NSAIDs include, without limitation, aceclofenac, acemetacin, actarit, alcofenac, alminoprofen, amfenac, aloxipirin, aminophenazone, antraphenine, aspirin, azapropazone, benorilate, benoxaprofen, benzydamine, butibufen, celecoxib, chlorthenoxacin, choline salicylate, clometacin, dexketoprofen, diclofenac, diflunisal, emorfazone, epirizole; etodolac, etoricoxib, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, lumiracoxib, mefenamic acid, meloxicam, metamizole, metiazinic acid, mofebutazone, mofezolac, nabumetone, naproxen, nifenazone, niflumic acid, oxametacin, phenacetin, pipebuzone, pranoprofen, propyphenazone, proquazone, protizinic acid, rofecoxib, salicylamide, salsalate, sulindac, suprofen, tiaramide, tinoridine, tolfenamic acid, valdecoxib, and zomepirac.

NSAIDs may be classified based on their chemical structure or mechanism of action. Non-limiting examples of NSAIDs include a salicylate derivative NSAID, a p-amino phenol derivative NSAID, a propionic acid derivative NSAID, an acetic acid derivative NSAID, an enolic acid derivative NSAID, a fenamic acid derivative NSAID, a non-selective cyclooxygenase (COX) inhibitor, a selective cyclooxygenase-1 (COX-1) inhibitor, and a selective cyclooxygenase-2 (COX-2) inhibitor. An NSAID may be a profen. Examples of a suitable salicylate derivative NSAID include, without limitation, acetylsalicylic acid (aspirin), diflunisal, and salsalate. Examples of a suitable p-amino phenol derivative NSAID include, without limitation, paracetamol and phenacetin. Examples of a suitable propionic acid derivative NSAID include, without limitation, alminoprofen, benoxaprofen, dexketoprofen, fenoprofen, flurbiprofen, ibuprofen, indoprofen, ketoprofen, loxoprofen, naproxen, oxaprozin, pranoprofen, and suprofen. Examples of a suitable acetic acid derivative NSAID include, without limitation, aceclofenac, acemetacin, actarit, alcofenac, amfenac, clometacin, diclofenac, etodolac, felbinac, fenclofenac, indometacin, ketorolac, metiazinic acid, mofezolac, nabumetone, naproxen, oxametacin, sulindac, and zomepirac. Examples of a suitable enolic acid (oxicam) derivative NSAID include, without limitation, droxicam, isoxicam, lornoxicam, meloxicam, piroxicam, and tenoxicam. Examples of a suitable fenamic acid derivative NSAID include, without limitation, flufenamic acid, mefenamic acid, meclofenamic acid, and tolfenamic acid. Examples of a suitable selective COX-2 inhibitors include, without limitation, celecoxib, etoricoxib, firocoxib, lumiracoxib, meloxicam, parecoxib, rofecoxib, and valdecoxib.

In some aspects, the cannabinoid analogs disclosed herein may be co-administered with other antibiotic(s) as part of a combination therapy. Combination therapy for suspected Gram-negative sepsis and severe infections with Pseudomonas may include a broad-spectrum beta-lactam and an aminoglycoside or a fluoroquinolone. Colistin combinations have been used as a last-resort treatment for multidrug-resistant strains. Combinations that include an aminoglycoside, ampicillin/sulbactam, a carbapenem, colistin, or rifampin have been successful against multidrug-resistant Acinetobacter. Colistin-tigecycline and other combinations including an aminoglycoside, a carbapenem, colistin, inhibitor, rifampin, or tigecycline have been studied for carbapenemase-producing Enterobacteriaceae. Combinations including a carbapenem have been suggested for these bacteria if the carbapenem minimum inhibitory concentration (MIC) is ≤4 mg/L. Non-limiting examples of antibiotics with which the cannabinoid analogs disclosed herein may be administered include amoxicillin, doxycycline, cephalexin, ciprofloxacin, clindamycin, metronidazole, azithromycin, sulfamethoxazole and trimethoprim. In some aspects, the cannabinoid analogs disclosed herein may exhibit, or may be co-administered with agents that exhibit anti-biofilm properties that may increase the efficacy of treatment and/or ameliorate the effects of antibiotic drug resistance.

In some aspects, a compound disclosed herein may be co-administered with a biofilm disruptor for treating a gram-negative bacterial infection. Peptides such as polymyxin B and the related colistin (polymyxin E) have been administered to humans as antibacterial agents and biofilm. However, their use has been previously limited because of their toxicity. These peptides comprise a seven amino acid cyclic peptide attached to an exocyclic three amino acid chain, wherein the N-terminal amine of the exocyclic chain is linked to an acyl side chain. By co-administering with a compound of the present disclosure, the biofilm disruptor may be administered at non-toxic dosages.

A therapeutically effective amount of a therapeutic compound disclosed herein generally is in the range of about 0.001 mg/kg/day to about 100 mg/kg/day. An effective amount may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In some examples, an effective amount of a therapeutic compound may be in the range of about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In other examples, an effective amount of a therapeutic compound disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day.

In addition to pharmaceutical compositions, compounds described herein may be formulated as an elixir, a beverage, a chew, a tablet, a lozenge, a gum, or the like. According to another aspect, the pharmaceutical compositions may also be formulated as a pharmaceutically acceptable vehicle such as a capsule, tablet, syrup, lozenge, inhaler, e-cigarette, chewable gum, nasal spray, transdermal patch, liquid, transmucosal vehicle, hydrogel, nanosome, liposome, noisome, nanoparticle, nanosphere, microsphere, microparticle, microemulsion, nanosuspension, or micelle. The compositions may also be formulated, for example, as dietary supplements or nutraceuticals.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### Example 1

This example illustrates the synthesis of (Z)-2-(3,6-dimethylhepta-2,5-dien-1-yl)-5-pentylbenzene-1,3-diol (“M194”). The compound was synthesized according the reaction sequence detailed below. The properties and characterization of the synthesis product are summarized in Table 1 below.

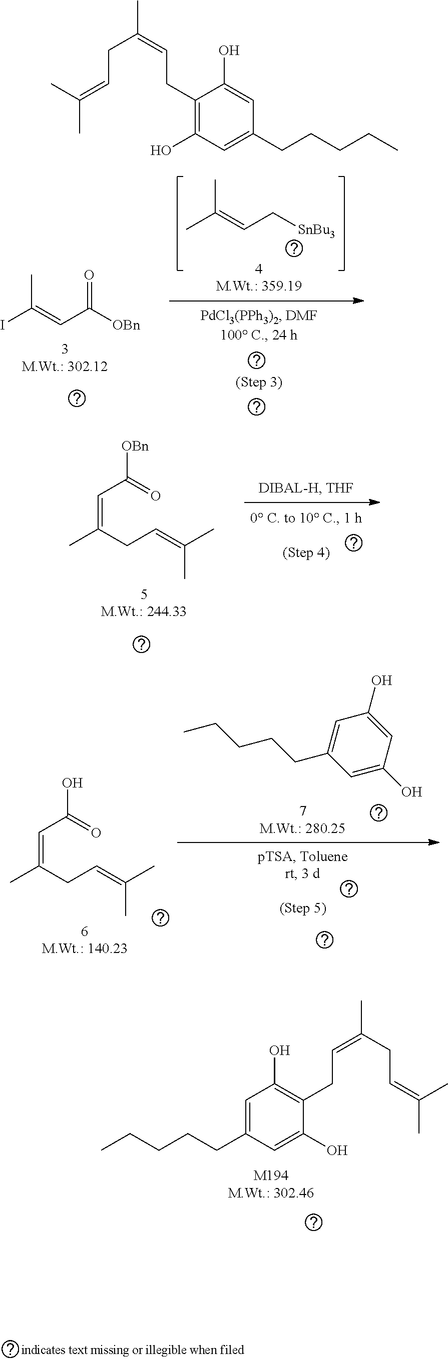

The starting material 3 in the above synthesis scheme was prepared as follows:

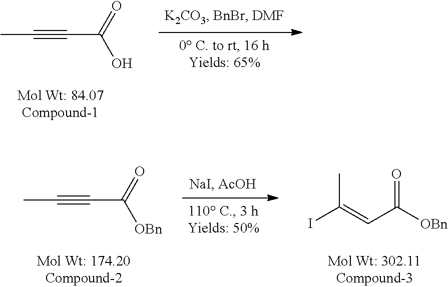

Reagent 4 in the above synthesis scheme was prepared as follows:

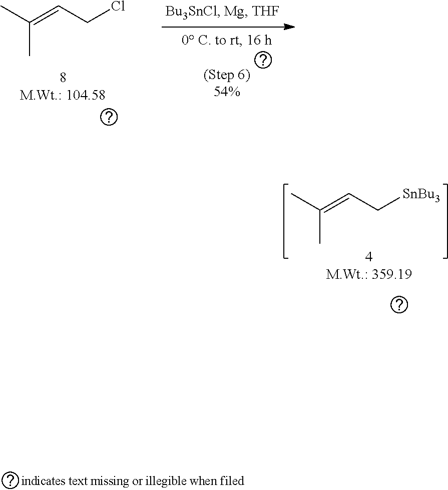

TABLE 1

| Test | Result |
|---|---|
| Appearance | Light Yellow Solid |
| NMR Spectrum | ¹H, 400 MHz, Dimethyl Sulfoxide-d₆, Consistent |
| Mass Spectrum | ESI, m/z 301.2 [M − H]⁻ |
| HPLC Analysis | >99% (area %), Kinetex 1.7 μmEv o C18 (2.1* |
| | 50 mm) Column, UV 215 nm Detection |

### Example 2

This example illustrates evaluating in vitro activity of (Z)-2-(3,6-dimethylhepta-2,5-dien-1-yl)-5-pentylbenzene-1,3-diol (Example 1, “M194”) against gram-positive and gram-negative bacteria. In this study, in vitro activity was determined against a diverse panel of Gram-positive and Gram-negative bacteria associated with a variety of infections. Broth microdilution MIC assays were conducted following guidelines recommended by the Clinical and Laboratory Standards Institute (CLSI; 1-3).

The compound M194 was provided in powder form and initially stored at room temperature prior to testing. A stock solution of M194 in DMSO/DMSO solvent/diluent was made at 101× the final testing concentration and tested over ranges of 0.06-64 μg/mL.

Test organisms consisted of isolates from the American Type Culture Collection (ATCC; Manassas, VA) and the Micromyx Repository (MMX; Kalamazoo, MI). Upon initial receipt at Microbiologics, the organisms were sub-cultured onto an appropriate agar medium and incubated under atmospheric conditions required for growth. Following incubation for 18 to 24 hr at 35° C. in the appropriate atmosphere, colonies were harvested from these plates and cell suspensions were prepared and frozen at −80° C. with a cryoprotectant. Prior to testing, isolates were streaked from frozen vials onto the appropriate agar. Unless otherwise noted, Trypticase Soy Agar with 5% sheep blood (Remel; Lenexa, KS; Lot No. 693244) was used and inoculated plates were incubated at 35° C. overnight in ambient atmosphere. Haemophilus influenzae isolates were streaked onto Chocolate agar (Becton Dickinson [BD]; Sparks, MD; Lot No. 3157581). Streptococcus spp., Moraxella spp., and H. influenzae inoculated plates were incubated at 35° C. overnight in 5% CO₂.

Organisms were tested in the appropriate media according to CLSI guidelines (1-3). Cation-Adjusted Mueller Hinton broth (CAMHB) was made by supplementing Mueller Hinton Broth (MHB; BD; Lot No. 1285575) to a final concentration of 20 mg/L Ca++ (Sigma; St. Louis, MO; Lot. No. 084K0215) and 12.5 mg/L Mg++ (VWR; Radnor, PA; Lot. No. 22C0856290). CAMHB was used for broth microdilution testing except for isolates requiring specialized media as described below.

CAMHB was supplemented with 5% lysed horse blood (LHB; Hemostat; Dixon, CA; Lot No. 711494) for testing of Streptococcus spp. and Listeria monocytogenes. For H. influenzae testing, Haemophilus Test medium (HTM) was made by supplementing CAMHB with 15 g/mL nicotinamide adenine dinucleotide (NAD; Sigma; Lot No. SLBX4629), 15 g/mL hematin porcine (Sigma; Lot No. SLCL9885), and 5 g/L of yeast extract (BD; Lot No. 7179576).

For broth microdilution testing of Neisseria gonorrhoeae, for which the reference CLSI methodology is agar dilution, ATCC Medium 814 was used to enable testing in broth. This medium contains 15 g Oxoid Special Peptone (Oxoid, Basingstoke, Hampshire England; Lot No. 2896869), 1 g corn starch (Ward's Science; Rochester, NY; Lot No. AD-13344-14), 5 g NaCl (VWR; Radnor, PA; Lot No. 57897), 4 g K2HPO4 (Sigma; Lot No. SLBW7134V) and 1 g KH2PO4 (Sigma; Lot No. 037K0132) per liter. After autoclaving, this medium was centrifuged at 5,000×g for 10 min and the supernatant filtered through a 0.45-micron sterile filter system. Following centrifugation and filtration, 1% IsoVitaleX enrichment (BD; Lot No. 3122835) was added.

### Broth Microdilution MIC Assay

The MIC assay method followed the procedures described by CLSI and employed automated liquid handlers to conduct serial dilutions and liquid transfers. Automated liquid handlers included the Multidrop Combi (ThermoScientific), Biomek 3000, and Biomek FX (Beckman Coulter; Fullerton, CA).

The wells in columns 2 through 12 in standard 96-well microdilution plates (Costar 3795) were filled with 150 μl of the appropriate diluent. These would become the “mother plates” from which “daughter,” or test plates, would be prepared. The drugs (300 μL at 101× the desired top concentration in the test plates) were dispensed into the appropriate well in column 1 of the mother plates. The Biomek 3000 was used to make serial two-fold dilutions through column 11 in the “mother plate.” The wells of column 12 contained no drug and served as the organism growth control wells.

Rows A through H of the daughter plates were loaded with 190 L per well of the appropriate test medium using the Multidrop Combi. The daughter plates were prepared using the Biomek FX which transferred 2 L of drug solution from each well of a mother plate to the corresponding well of the daughter plate in a single step. A standardized inoculum of each organism was prepared per CLSI (1, 3). Colonies were picked from the streak plate and a suspension was prepared to equal a 0.5 McFarland turbidity standard in saline. Suspensions were then diluted 1:20 and transferred to compartments of sterile reservoirs. Daughter plates were placed on the Biomek 3000 in reverse orientation so that plates were inoculated from low to high drug concentration. The Biomek 3000 delivered 10 L of standardized inoculum into each well of the appropriate daughter plate for an additional 1:20 dilution, targeting a final concentration of 5×105 CFU/mL.

Inoculated plates were covered with a sterile lid, placed in plastic bags, and were incubated at 35° C. under ambient atmosphere for 20 hr. After incubation, plates were viewed from the bottom using a plate viewer. An un-inoculated solubility control plate was observed for evidence of drug precipitation. MIC values were read where visible growth of the organism was inhibited.

The in vitro activity of M194 was determined against Gram-positive and Gram-negative bacteria associated with a variety of infections. The results from broth microdilution testing are presented in Table 2 and Table 3, for Gram-positive and Gram-negative isolates, respectively.

### Gram-Positive Isolates

M194 had activity against all Staphylococcus spp., Enterococcus spp., and to a lesser degree, against all Streptococcus spp. For Staphylococcus spp. and Enterococcus spp., the MIC values ranged from 1 to 2 g/mL, and ranged from 8-32 g/mL for Streptococcus spp. and L. monocytogenes. The observed activity was consistent across isolates with varied susceptibility profiles including methicillin-resistant S. aureus (MRSA), vancomycin-intermediate S. aureus (VISA), vancomycin-resistant Enterococcus (VRE), and multi-drug resistant (MDR) Streptococcus pneumoniae. The controls levofloxacin, tobramycin, ampicillin, tetracycline, tigecycline, daptomycin, and vancomycin behaved as expected against these isolates.

TABLE 2

| | | | MIC |
|---|---|---|---|
| Organism | | Type | (μg/mL) |
| Coagulase-positive | Staphylococcus aureus | MSSA; QC | 2 |
| Staphylococcus | | MSSA | 1 |
| | | MRSA | 1 |
| | | VISA | 2 |
| Coagulase-negative | Staphylococcus | MSSE | 1 |
| Staphylococcus | epidermidis | MRSE | 1 |
| | Staphylococcus | MSS | 2 |
| | saprophyticus | | |
| — | Enterococcus faecalis | VSE; QC | 2 |
| | | VSE | 1 |
| | | vanA | 1 |
| | | vanB | 1 |
| | Enterococcus faecium | — | 1 |
| | | vanA | 1 |
| β-hemolytic | Streptococcus pyogenes | — | 16 |
| Streptococci | | ERYR | 16 |
| | Streptococcus agalactiae | — | 16 |
| | | MLS | 16 |
| Viridans | Streptococcus constellatus | — | 16 |
| Streptococci | Streptococcus oralis | ERYR | 32 |
| | Streptococcus mutans | — | 32 |
| — | Listeria monocytogenes | — | 32 |

MIC: minimum inhibitory concentration, QC: quality control, VAN: vancomycin, MSSA: methicillin-susceptible S. aureus, MRSA: methicillin-resistant S. aureus, VISA: vancomycin-intermediate S. aureus, MSSE: methicillin-susceptible S. epidermidis, MRSE: methicillin-resistant S. epidermidis, VRE: vancomycin-resistant Enterococcus, VSE: vancomycin-susceptible Enterococcus, PISP: penicillin-intermediate S. pneumoniae, PRSP: penicillin-resistant S. pneumoniae, MDR: multidrug-resistant, MLS: macrolide-, lincosamide-, streptogramin-resistant, ERYR: erythromycin-resistant, ESBL: extended-spectrum beta-lactamase positive, NDM-1: New Delhi metallo-beta-lactamase positive, KPC-2: Klebsiella pneumoniae carbapenemase positive, VIM: Verona integron-encoded metallo-beta-lactamase positive, BL+: beta-lactamase positive.

### Gram-Negative Isolates

M194 had activity against H. influenzae, Moraxella catarrhalis, and N. gonorrhoeae. M194 was not active against, P. aeruginosa, Acinetobacter baumannii, or any of the Enterobacterales. Against H. influenzae, M194 was slightly less active against the one beta-lactamase positive isolate tested (MMX 7988) with an MIC value of 32 μg/mL relative to the ATCC 49247 isolate that had an MIC value of 4 μg/mL. The lowest MIC values for M194 against Gram-negative isolates were observed against M. catarrhalis with an MIC value of 2 μg/mL and against N. gonorrhoeae with an MIC value of 1 μg/mL. The controls levofloxacin, tobramycin, ampicillin, tetracycline, tigecycline, meropenem, and colistin behaved as expected against these isolates.

TABLE 3

| | | | MIC |
|---|---|---|---|
| Organism | | Type | (μg/mL) |
| Enterobacterales | Escherichia coli | QC | >64 |
| | | ESBL | >64 |
| | | NDM-1 | >64 |
| | Klebsiella pneumoniae | QC | >64 |
| | | — | >64 |
| | Enterobacter cloacae | — | >64 |
| | Citrobacter freundii | — | >64 |
| | Proteus mirabilis | — | >64 |
| | Serratia marcescens | — | >64 |
| Other Gram- | Pseudomonas aeruginosa | QC | >64 |
| negatives | | MDR; VIM | >64 |
| | Acinetobacter baumannii | — | >64 |
| | | MDR | >64 |
| | Haemophilus influenzae | QC | 4 |
| | | BL+ | 32 |
| | Moraxella catarrhalis | BL+ | 2 |
| | Neisseria gonorrhoeae | QC | 1 |

MIC: minimum inhibitory concentration, QC: quality control, MDR: multidrug-resistant, ESBL: extended-spectrum beta-lactamase positive, NDM-1: New Delhi metallo-beta-lactamase positive, KPC-2: Klebsiella pneumoniae carbapenemase positive, VIM: Verona integron-encoded metallo-beta-lactamase positive, BL+: beta-lactamase positive.

While the invention has been described with respect to specific examples, those skilled in the art will appreciate that there are numerous variations and permutations of the above described systems and techniques that fall within the spirit and scope of the invention as set forth in the appended claims.

1.-5. (canceled)

6. A compound having a structure of Formula (VI): 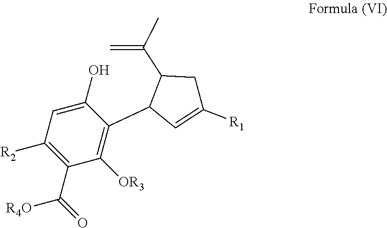
wherein R₁, R₂, R₃ and R₄ are each independently selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COH, —C(O)—C₁₋₄ alkyl, —C(O)O C₁₋₄ alkyl, —NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt, ester or ether thereof.

7. A pharmaceutical composition comprising a therapeutically effective amount of a compound of claim 6 and a pharmaceutically acceptable vehicle therefor.

8. A method of treating a bacterial infection comprising administering to an individual in need thereof a pharmaceutical composition of claim 7.

9. A method of treating anxiety, depression or pain comprising administering to an individual in need thereof a pharmaceutical composition of claim 7.

10. (canceled)

11. A compound of claim 6 which has a structure selected from the group consisting of: 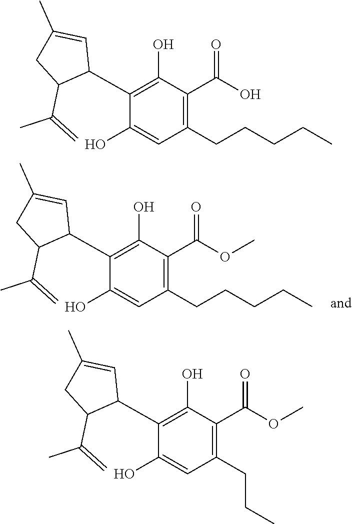
or a pharmaceutically acceptable salt, ester or ether thereof.

12. A pharmaceutical composition comprising a therapeutically effective amount of a compound of claim 11 and a pharmaceutically acceptable vehicle therefor.

13. A method of treating a bacterial infection comprising administering to an individual in need thereof a pharmaceutical composition of claim 12.

14. The method of claim 13, wherein the bacterial infection is a methicillin-resistant Staphylococcus aureus (MRSA) infection.

15. The method of claim 13, wherein the pharmaceutical composition is administered to an individual who has been exposed or is at risk of exposure to Bacillus anthracis or Bacillus anthracis spores.

16.-20. (canceled)

21. The compound of claim 6, wherein R₁ is CH₃, R₃ is H, and R₄ is H or CH₃ alkyl.

22. The compound of claim 21, wherein R₂ is C₃₋₇ alkyl.

23. A pharmaceutical composition comprising a therapeutically effective amount of a compound of claim 22 and a pharmaceutically acceptable vehicle therefor.

24. A method of treating a bacterial infection comprising administering to an individual in need thereof a pharmaceutical composition of claim 23.

25. A compound having a structure of Formula (VII): 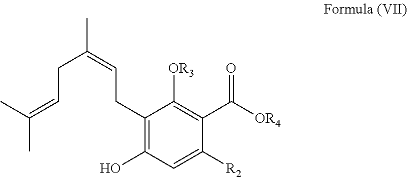
wherein R₂, R₃ and R₄ are each independently selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt, ester or ether thereof.

26. The compound of claim 25, wherein R₃ is H, and R₄ is H or C₁₋₃ alkyl.

27. The compound of claim 26, wherein R₂ is C₃₋₇ alkyl.

28. A pharmaceutical composition comprising a therapeutically effective amount of a compound of claim 27 and a pharmaceutically acceptable vehicle therefor.

29. A method of treating a bacterial infection comprising administering to an individual in need thereof a pharmaceutical composition of claim 28.

30. The method of claim 29, wherein the pharmaceutical composition is administered to an individual who has been exposed or is at risk of exposure to Bacillus anthracis or Bacillus anthracis spores.

31. A compound having a structure selected from the group consisting of: 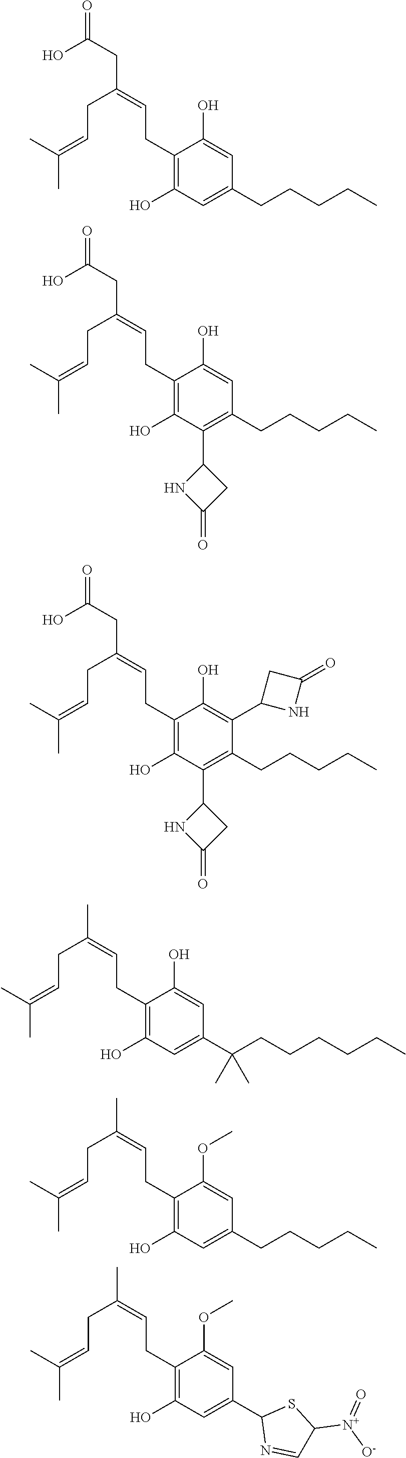 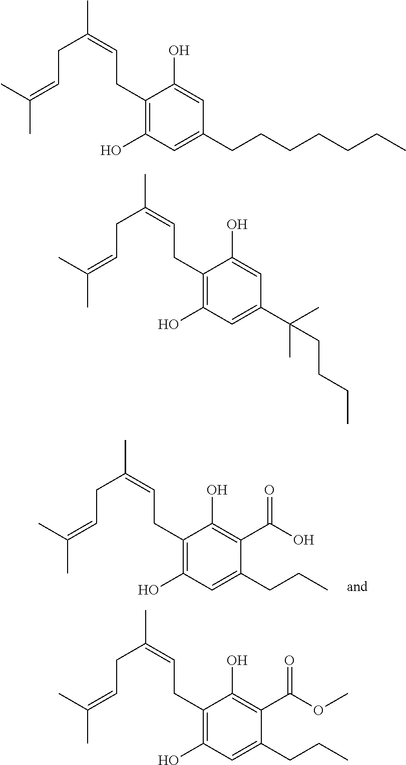
or a pharmaceutically acceptable salt, ester or ether thereof.

32. A method of treating a bacterial infection comprising administering to an individual in need thereof a therapeutically effective amount of a compound of Formula (I): 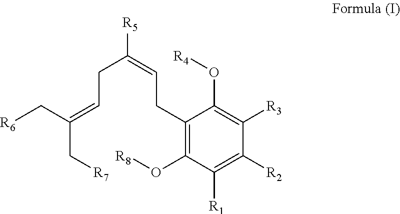
wherein R₁, R₂, R₃, R₄, R₅, R₆, R₇, and R₈ are each independently selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, OH, alkyl, —O-alkyl, —COH, —C(O)—C₁₋₄ alkyl, —C(O)O—C₁₋₄ alkyl, —NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt, ester or ether thereof.

33. The method of claim 32, wherein the bacterial infection is a methicillin-resistant Staphylococcus aureus (MRSA) infection.

34. The method of claim 32, wherein the individual has been exposed or is at risk of exposure to Bacillus anthracis or Bacillus anthracis spores.

35. The method of claim 32, wherein the compound has the structure: 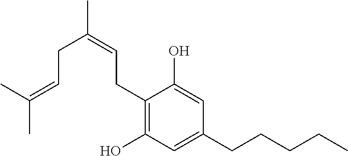 [SMILES: CCCCCc1cc(O)c(C/C=C(/C)CC=C(C)C)c(O)c1]
or a pharmaceutically acceptable salt, ester or ether thereof.

36. A method of treating a viral infection comprising administering to an individual in need thereof a therapeutically effective amount of a compound of Formula (I): 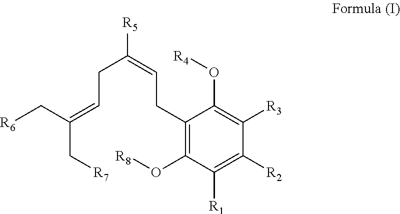
wherein R₁, R₂, R₃, R₄, R₅, R₆, R₇, and R₈ are each independently selected from the group consisting of H, OH, protected hydroxyl, alkyl, alkenyl, alkynyl, acyl, aryl, heteroaryl, cycloalkyl, and heterocycle; wherein the alkyl, alkenyl, alkynyl or acyl is optionally substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, OH, alkyl, —O-alkyl, —COH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt, ester or ether thereof.

37. The method of claim 36, wherein the viral infection is from a virus selected from the group consisting of a coronavirus, hepatitis, HIV, and influenza.

38. The method of claim 36, wherein the compound has the structure: 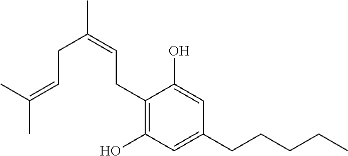 [SMILES: CCCCCc1cc(O)c(C/C=C(/C)CC=C(C)C)c(O)c1]
or a pharmaceutically acceptable salt, ester or ether thereof.
